(19) United States
(12) Patent Application Publication
(10) Pub. No.: US 2004/0102375 A1
(43) Pub. Date: May 27, 2004

(54) Methods to overcome inhibition of growth cone translocation

(75) Inventors: Lorene M. Lanier, St. Paul, MN (US); Frank B. Gertler, Boston, MA (US)

Correspondence Address: Helen C, Lockhart, Wolf, Greenfield & Sacks, P.C., Federal Reserve Plaza, 600 Altantic Avenue, Boston, MA, 02210, US

(73) Assignee: Massachusetts Institute of Technology, Cambridge, MA

(21) Appl. No.: 10/439,276

(22) Filed: May 15, 2003

Related U.S. Application Data
(60) Provisional application No. 60/380,597, filed on May 15, 2002.

(51) Int. Cl. A61K 38/17

(52) U.S. Cl. 514/12

(57) ABSTRACT

The invention relates to methods for regulating neural growth and regeneration. In particular methods for promoting neural growth are described herein. The methods have a variety of clinical, diagnostic and therapeutic uses.

### RELATED APPLICATIONS

This application claims priority to U.S. Provisional Patent Application No. 60/380,597 filed May 15, 2002, the entire contents of which is hereby incorporated by reference.

### GOVERNMENT SUPPORT

The present invention was supported in part by a grant from the United States National Institutes of General Medical Science under contract/grant number GM58801. The U.S. Government may retain certain rights in the invention.

### FIELD OF THE INVENTION

The invention relates to methods for regulating neural growth and regeneration and related products. In particular methods for promoting neural regeneration, overcoming neuronal inhibitory signals, and for preventing nerve degeneration are described herein.

### BACKGROUND OF THE INVENTION

Injury to the nervous system may result in irreversible functional impairments. Nerve cells that are lost may not be replaced. Those that are spared are sometimes unable to regrow severed connections. A limited amount of local synaptic reorganization, however, can occur close to the site of injury. Within the brain or spinal cord, damage resulting from stroke, trauma, or other causes can result in serious loss of cognitive, sensory and motor functions.

The inability of the nervous system to regenerate after injury has been attributed to a number of factors, which include the presence of inhibitory molecules that suppress axonal growth; absence of activating molecules which foster growth; and an absence of the appropriate trophic factors needed to activate gene expression required for cell survival and differentiation. Even when the activating molecules and appropriate trophic factors are added, generally nerve growth cannot be improved because of the presence of the inhibitory signals.

### SUMMARY OF THE INVENTION

The invention relates, in some aspects, to methods for promoting neural growth and regeneration and for various therapeutic treatments using Arp2/3 inhibitors. It has been discovered, surprisingly, that Arp2/3 may actually be a negative regulator of growth cone translocation. Studies involving fibroblasts had previously established a positive role for Arp2/3 in the regulation of growth and motility. It was discovered herein that inhibitors of Arp2/3 are able to overcome the inhibition of growth cone translocation, indicating that Arp2/3 actually functions as a negative regulator in neural cells.

In one aspect the invention is a method for enhancing nerve cell growth by reducing Arp2/3 activity in a nerve cell in an effective amount to enhance nerve cell growth. In one embodiment the Arp2/3 activity is reduced by contacting the nerve cell with an Arp2/3 inhibitor. An Arp2/3 inhibitor may be, for example, an Arp2/3 binding molecule that interacts with and reduces the activity of an Arp2/3 complex, an Arp2/3 antisense molecule or an Arp2/3 RNAi molecule. In some embodiments the Arp2/3 binding molecule is an acidic domain of N-WASP or a functionally active fragment thereof. In other embodiments the Arp2/3 binding molecule is an acidic domain of WASP, WAVE or Scar or a functionally active fragment thereof. In yet other embodiments the Arp2/3 binding molecule is a peptide mimetic.

The method may be an in vitro method or an in vivo method, e.g. a method for promoting neural regeneration in vivo.

In other aspects, a method for identifying a therapeutic nerve growth promoter is provided. The method involves identifying a molecule capable of binding to Arp2/3 to identify a putative Arp2/3 binding molecule, contacting a nerve cell with the putative Arp2/3 binding molecule, and determining the effect of the putative Arp2/3 binding molecule on cell growth cone motility, wherein the putative Arp2/3 binding molecule is a therapeutic nerve growth promoter when the nerve cell demonstrates enhanced motility with respect to a nerve cell that has not been contacted with the putative Arp2/3 binding molecule.

A method for overcoming neuronal inhibitory signals, is provided according to other aspects. The method involves contacting a nerve cell with an Arp2/3 inhibitor in an effective amount to overcome an inhibitory factor in the nerve cell.

A method for promoting nerve generation by contacting nerve cells with an Arp2/3 inhibitor in an effective amount to promote nerve generation is provided according to other aspects. In one embodiment the Arp2/3 inhibitor is administered to a site of damaged nerve cells in a subject.

According to other aspects a method for treating neurodegeneration is provided. The method involves administering to a subject having or at risk of neurodegeneration an Arp2/3 inhibitor in an amount effective to treat neurodegeneration.

The Arp2/3 inhibitor may be administered locally to the site of neurodegeneration, optionally in a sustained release vehicle. In other embodiments the Arp2/3 inhibitor is administered to a nerve cell in vitro and the nerve cell is delivered to the subject at the site of neurodegeneration.

The subject having or at risk of neurodegeneration may have or may be at risk of developing Alzheimer's disease, Down Syndrome; Parkinson's disease; ainyotrophic lateral sclerosis (ALS), stroke, direct trauma, Huntington's disease, epilepsy, ALS-Parkinsonism-dementia complex; progressive supranuclear palsy; progressive bulbar palsy, spinomuscular atrophy, cerebral amyloidosis, Pick's atrophy, Retts syndrome; Wilson's disease, Striatonigral degeneration, corticobasal ganglionic degeneration; dentatoruibral atrophy, olivo-pontocerebellar atrophy, paraneoplastic cerebellar degeneration; Tourettes syndrome, hypoglycemia; hypoxia; CreLitzfeldt-Jakob disease; or Korsakoff's syndrome.

Each of the embodiments of the invention can encompass various recitations made herein. It is, therefore, anticipated that each of the recitations of the invention involving any one element or combinations of elements can, optionally, be included in each aspect of the invention.

### BRIEF DESCRIPTION OF THE DRAWINGS

FIG. 1: Immunofluorescence analysis depicting localization of Arp2/3 in fibroblasts and neuronal cells.

FIG. 2: Immunofluorescence analysis depicting neurons expressing the acidic domain of N-WASP (right panel labeled +N-WASP(A)) or a control vector (left panel labeled +EGFP). The neurons expressing the acidic domain of N-WASP have longer, more branched neurites.

FIG. 3A and FIG. 3B: Immunofluorescence analysis depicting neurons expressing a control vector (EGFP) grown on a coverslip having sections coated with semaphorin 3A (labeled +) or uncoated (labeled −). The neurons expressing the control vector have very few or no neurites which extend onto the semaphorin 3A coated section of the coverslip.

FIG. 4A and FIG. 4B: Immunofluorescence analysis depicting neurons expressing acidic domain of N-WASP (EGFP-NWA) grown on a coverslip having sections coated with semaphorin 3A (labeled +) or uncoated (labeled −). The neurons expressing the acidic domain of N-WASP produce long neurites which extend over both the coated and uncoated section of the coverslip.

### BRIEF DESCRIPTION OF THE SEQUENCES

SEQ. ID. NO. 1 is the amino acid sequence of the acidic domain of N-WASP: VGALMEVMQKRSKAIHSSDEDEDEDDDEDFEDDDEWED.

SEQ. ID. NO. 2 is the amino acid sequence of the acidic domain of WASP: VGALMEVMQKRSKAIHSSDEGEDQAGDEDEDDDEWDD.

SEQ. ID. NO. 3 is the amino acid sequence of the acidic domain of Scar/WAVE: IENDVATILSRRIAVEYSDSEDDSEFDEVDWLE.

### DETAILED DESCRIPTION

The invention is based in part on the surprising discovery that Arp2/3 is a negative regulator of neuronal growth cone translocation. Arp2/3 has been established to be a positive regulator of cell motility in fibroblasts. In view of the finding that Arp2/3 is a negative regulator of nerve cell growth, therapeutic methods for overcoming inhibition of growth cone translocation can now be accomplished. This is useful for a variety of research, diagnostic and therapeutic purposes. For instance, these inhibitors may be used to enhance nerve cell growth by reducing Arp2/3 activity in a nerve cell, and thus are useful in the process of nerve regeneration in subjects having experienced nerve injury. The findings described herein are also useful for identifying other therapeutic compounds useful in this process.

Enhancing “nerve cell growth” as used herein refers to any increase in growth cone translocation. This property can be assessed using a variety of assays known in the art. For instance, a nerve can be directly examined using immunofluorescence in the presence or absence of the treatment. A nerve which has longer neurites (including axons and/or dendrites) in the presence of the treatment compared to “prior to treatment” or compared to a control is one in which nerve cell growth has been enhanced.

The methods are accomplished using an Arp2/3 inhibitor. An “Arp2/3 inhibitor” as used herein is any compound which prevents the activity of Arp2/3 protein complex. Arp2/3 inhibitors include but are not limited to an Arp2/3 binding molecules that interact with and reduce the activity of Arp2/3 complex, Arp2/3 antisense molecules, Arp2/3 RNAi, and Arp2/3 dominant negative proteins.

An “Arp2/3 binding molecule” as used herein is any type of molecule that specifically binds to Arp2/3 and reduces its activity. A molecule reduces Arp2/3 activity when it causes a decrease or at least partially interferes with the activity of the complex when the complex is exposed to the binding molecule compared to an Arp2/3 complex not exposed to such a molecule. Arp2/3 is known to bind to acidic domains of proteins such as N-WASP, WASP, and Scar/WAVE. These acidic domains inhibit the activity of the Arp2/3 complex. Other types of Arp2/3 binding molecules include antibodies, antibody fragments, other peptides, mimetics, etc. Other Arp2/3 binding molecules can be identified using routine binding assays. The ability of a molecule that binds to Arp2/3 to reduce the activity of Arp2/3 can be assessed using assays such as those described herein.

The WASP family of proteins includes at least WASP, N-WASP and Scar/WAVE. The members of this family of proteins all include an acidic domain that has a high degree of homology. The acidic domain of these family members have the following amino acid sequences:

| | | |
|---|---|---|
| | 10 20 30 | |
| N-WASP | VGALMEVMQKRSKAIHSSDEDEDEDDDEDFEDDDEWED | (SEQ ID NO. 1) |
| WASP | VGALMEVMQKRSKAIHSSDEGEDQAGDED-EDDDEWDD | (SEQ ID NO. 2) |
| Scar/WAVE | IENDVATILSRRIAVEYSDS-ED---DSE-FDEVDWLE | (SEQ ID NO. 3) |
| | . . . * *. ** ** * . *. .* . | |

The acidic domain is known to bind to and inhibit the activity of Arp2/3. These proteins have been described in a number of references, including a review article by Mullins, R D (Current Opinion in Cell Biology, 2000, 12:91-96).

Thus, the Arp2/3 binding molecules include the acidic domains of the WASP family of proteins and functional equivalents thereof. The acidic domain of the WASP protein, or functional variants or fragments thereof may be delivered directly to nerve cells in vitro or to a subject in vivo as a peptide or in a nucleic acid vector which will express the peptide in vivo. Preferably the peptides, when delivered in vivo are isolated. As used herein with respect to peptides, “isolated” means separate from its native environment. Isolated, when referring to a protein or peptide, means, for example: (i) selectively produced by expression cloning or (ii) purified as by chromatography or electrophoresis. Isolated proteins or peptides may, but need not be, substantially pure. The term “substantially pure” means that the proteins or peptides are essentially free of other substances with which they may be found in nature or in vivo systems to an extent practical and appropriate for their intended use. Substantially pure peptides may be produced by techniques well known in the art. Because an isolated protein may be admixed with a pharmaceutically acceptable carrier in a pharmaceutical preparation, the peptide may comprise only a small percentage by weight of the preparation. The peptide is nonetheless isolated in that it has been separated from the substances with which it may be associated in living systems, i.e. isolated from other peptides.

As used herein, a “variant” of an acidic domain of a WASP protein is a peptide which contains one or more modifications to the primary amino acid sequence of the acidic domain of a WASP protein, but which is still capable of reducing Arp2/3 activity. Conservative amino acid substitutions may be made in WASP family of proteins to provide functionally equivalent variants of the foregoing peptides, i.e., the variants retain the functional capabilities of the these proteins. As used herein, a “conservative amino acid substitution” refers to an amino acid substitution which does not alter the relative charge or size characteristics of the protein in which the amino acid substitution is made. Variants can be prepared according to methods for altering peptide sequence known to one of ordinary skill in the art such as are found in references which compile such methods, e.g. Molecular Cloning: A Laboratory Manual, J. Sam brook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of the acidic domain of the WASP proteins include conservative amino acid substitutions of the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Conservative amino-acid substitutions in the amino acid sequence of WASP proteins to produce functionally equivalent variants of WASP proteins typically are made by alteration of a nucleic acid encoding a WASP protein. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding an acidic domain of a WASP protein. The activity of functionally equivalent fragments of WASP proteins can be tested by cloning the gene encoding the altered WASP protein into a bacterial or mammalian expression vector, introducing the vector into an appropriate host cell, expressing the altered WASP protein, and testing for a functional capability of the WASP proteins as disclosed herein.

The Arp2/3 binding molecules that function as inhibitors also embrace peptide and non-peptide binding agents, in addition to the acidic domains of the WASP family of proteins and functional equivalents thereof which, for example, can be peptide mimetics, peptides, antibodies or fragments of antibodies having the ability to selectively bind to Arp2/3 proteins and reduce its activity.

Thus, the invention involves peptides of numerous size and type that bind specifically to Arp2/3 proteins. These peptides may be derived from a variety of sources. For example, such peptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention, including human antibodies. Briefly, one prepares a phage library (using e.g. ml3, fd, or lambda phage), displaying inserts from 4 to about 80 amino acid residues using conventional procedures. The inserts may represent, for example, a completely degenerate or biased array. One then can select phage-bearing inserts which bind to the Arp2/3 protein complex. This process can be repeated through several cycles of reselection of phage that bind to the Arp2/3. Repeated rounds lead to enrichment of phage bearing particular sequences. DNA sequence analysis can be conducted to identify the sequences of the expressed peptides. The minimal linear portion of the sequence that binds to the Arp2/3 can be determined. One can repeat the procedure using a biased library containing inserts containing part or all of the minimal linear portion plus one or more additional degenerate residues upstream or downstream thereof. Yeast two-hybrid screening methods also may be used to identify peptides that bind to the Arp2/3 protein complex. Thus, the Arp2/3 complex, or a fragment thereof, can be used to screen peptide libraries, including phage display libraries, to identify and select peptide binding partners of the Arp2/3.

The crystal structure of Arp2/3 has been elucidated and described in the literature, e.g., see Welsh, and Borths (Structure, 2002, 10:131-135.). The types of structures which bind to Arp2/3 complex is also known. For instance, as described above, it is known that the acidic domain of the WASP family of proteins bind to Arp2/3.

Based on this information peptide and non-peptide libraries which are based on the known Arp2/3 binding proteins can easily be generated by those of skill in the art. Commercial entities such as ArQule (Woburn, Mass.) prepare custom libraries for the generation of mimetic compounds. The Arp2/3 binding compounds or putative binding compounds in such libraries may be identified using any of the screening assays or methods described herein.

Antibodies may also be useful as Arp2/3 binding molecules. Antibodies include polyclonal and monoclonal antibodies, as well as antibody fragments prepared according to conventional methodology. Only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fe region has been enzymatically cleaved, or which has been produced without the Fe region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

As mentioned above, the Arp2/3 inhibitors embrace antisense oligonucleotides that selectively bind to an Arp2/3 nucleic acid molecule, to reduce the expression of Arp2/3. As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions. Based upon the sequences of nucleic acids encoding Arp2/3 proteins, or upon allelic or homologous genomic and/or cDNA sequences, one of skill in the art can easily choose and synthesize any of a number of appropriate antisense molecules for use in accordance with the present invention.

In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind. The Arp2/3 binding molecules encompass antisense oligonucleotides which are complementary to the cDNA or genomic DNA corresponding to nucleic acids encoding Arp2/3 proteins.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, nucleic acids encoding Arp2/3 proteins, together with pharmaceutically acceptable carriers.

Antisense oligonucleotides may be administered as part of a pharmaceutical composition. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient.

In another embodiment, activity of the Arp2/3 protein can be reduced by specifically down-regulating the expression of the Arp2/3 protein using double-stranded RNA inhibition (RNAi). The double-stranded Arp2/3 RNA should be capable of selectively inhibiting expression of the endogenous Arp 2 and/or 3 to prevent formation of a functional Arp2/3 protein. RNAi technology is well known in the art as a tool for inhibiting expression of a specific target gene e.g., see Fire et al., Nature 391:801-811 (1998) and Timmins and Fire, Nature 395:854 (1998). The standard approach is based on delivery or injection of dsRNA directly into the cell or organism.

The invention also provides, in certain embodiments, “dominant negative” peptides derived from Arp2/3 proteins. A dominant negative peptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative Arp2/3 peptide in a cell is a reduction in function of active Arp2/3 proteins and/or assembly of a functional complex. One of ordinary skill in the art can assess the potential for a dominant negative variant of a protein, and using standard mutagenesis techniques to create one or more dominant negative variant peptides. For example, given the teachings contained herein of Arp2/3 proteins, one of ordinary skill in the art can modify the sequence of the Arp2/3 proteins by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. Other similar methods for creating and testing dominant negative variants of a protein will be apparent to one of ordinary skill in the art.

In addition to the delivery of peptides which are Arp2/3 binding molecules, nucleic acid vectors encoding such peptides may be administered to a cell or a subject so that the peptide can be expressed in the nerve cell. When nucleic acids encoding the Arp2/3 binding molecules are delivered to the cell it is generally within an expression vector in order to produce functional peptides within the cells.

The nucleic acids useful for delivery to the cell include nucleic acid coding sequences operably joined to expression sequences, optionally in a vector. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single the per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (I) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or peptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989.

Preferred systems for mRNA expression in mammalian cells are those such as pcDNA3.1 and pRc/CMV (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein-Barr Virus (EBV) origin of replication, facilitating the maintenance of plasmid as a multicopy extrachromosomal element. Another expression vector is the pEF-BOS plasmid containing the promoter of peptide Elongation Factor Iα, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.PIA recombinant for the expression of an antigen is disclosed by Warnier et al., in intradermal injection in mice for immunization against PlA (Int. J. Cancer, 67:303-310, 1996). Additional vectors for delivery of nucleic acid are provided below.

The inhibitors are useful for treating nerve disorders, such as diseases associated with neurodegeneration and injuries resulting in nerve damage, in which nerve regeneration may be desirable. The terms “treat” and “treating” as used herein refer to partially or completely promoting growth cone translocation, motility or migration in a nerve cell, as well as, inhibiting any increase in inhibition of growth cone translocation which would otherwise exacerbate the medical condition. Thus, a subject is treated if any increase in growth cone translocation, motility or migration in a nerve cell is observed.

The nerve cells may be treated in vivo, in vitro, or ex vivo. Thus, the cells may be in an intact subject or isolated from a subject or alternatively may be an in vitro cell line. A “subject” as used herein refers to a human or non-human mammal including but not limited to primates, dogs, cats, horses, sheep, goats, cows, rabbits, pigs and rodents.

The terms “translocation”, “growth”, “motility” or “migration” when used with respect to a nerve cell or neural growth cone, are used synonymously to refer to neurite extension or growth. Neurite extension or growth may result in an axon and/or a dendrite.

The discovery that nerve cell growth can be induced by decreasing the level of functional Arp2/3 complex in a cell has important implications for regeneration of nerve tissue, including, for instance neuro-regeneration therapies, or treatment of neurodegenerative disease.

Thus the invention contemplates the treatment of subjects having or at risk of developing neurodegenerative disease or an injury to nerve cells in order to cause neuro-regeneration. Neuronal cells are predominantly categorized based on their local/regional synaptic connections (e.g., local circuit interneurons vs. longrange projection neurons) and receptor sets, and associated second messenger systems. Neuronal cells include both central nervous system (CNS) neurons and peripheral nervous system (PNS) neurons. There are many different neuronal cell types. Examples include, but are not limited to, sensory and sympathetic neurons, cholinergic neurons, dorsal root ganglion neurons, proprioceptive neurons (in the trigeminal mesencephalic nucleus), ciliary ganglion neurons (in the parasympathetic nervous system), etc. A person of ordinary skill in the art will be able to easily identify neuronal cells and distinguish them from non-neuronal cells such as glial cells, typically utilizing cell-morphological characteristics, expression of cell-specific markers, secretion of certain molecules, etc. Arp2/3 proteins have been identified in neuronal growth cones.

“Neurodegenerative disorder” is defined herein as a disorder in which progressive loss of neurons occurs either in the peripheral nervous system or in the central nervous system. Examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapic, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor). and Wernicke-Korsakoff's related dementia. Neurodegenierative diseases affecting sensory neurons include Friedreich's ataxia, diabetes, peripheral neuropathy, and retinal neuronal degeneration. Neurodegenerative diseases of limbic and cortical systems include cerebral amyloidosis, Pick's atrophy, and Retts syndrome. The foregoing examples are not meant to be comprehensive but serve merely as an illustration of the tern “neurodegenerative disorder.”

Most of the chronic neurodegenerative diseases are typified by onset during the middle adult years and lead to rapid degeneration of specific subsets of neurons within the neural system, ultimately resulting in premature death. The Arp2/3 inhibitor may be administered to a subject to treat or prevent neurodegenerative disease or to promote tissue generation alone or in combination with the administration of other therapeutic compounds for the treatment or prevention of these disorders or promotion of tissue generation. May of these drugs are known in the art.

For example, antiparkinsonian agents include but are not limited to Benztropine Mesylate; Biperiden; Biperiden Hydrochloride; Biperiden Lactate; Carmantadiine; Ciladopa Hydrochloride; Dopamantine; Ethopropazine Hydrochloride; Lazabemide; Levodopa; Lometraline Hydrochloride; Mofegiline Hydrochloride; Naxagolide Hydrochloride; Pareptide Sulfate; Procyclidine Hydrochloride; Quinelorane Hydrochloride; Ropinirole Hydrochloride; Selegiline Hydrochloride; Tolcapone; Trihexyphenidyl Hydrochloride. Drugs for the treatment of amyotrophic lateral sclerosis include but are not limited to Riluzole. Drugs for the treatment of Paget's disease include but are not limited to Tiludronate Disodium.

When administered, the therapeutic compositions of the present invention can be administered in pharmaceutically acceptable preparations. Such preparations may routinely contain pharmaceutically acceptable concentrations of salt, buffering agents, preservatives, compatible carriers, and optionally other therapeutic agents.

The therapeutics of the invention can be administered by any conventional route, including injection or by gradual infusion over time. The administration may, for example, be oral, pulmonary, respiratory, nasal, intravenous, intraperitoneal, mucosal (i.e. rectal, vaginal, ocular), intramuscular, intracavity, subcutaneous, or transdermal. Techniques for preparing aerosol delivery systems containing active agents are well known to those of skill in the art. Generally, such systems should utilize components which will not significantly impair the biological properties of the active agents (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation. When using antisense preparations, intravenous or oral administration are preferred.

The compositions of the invention are administered in effective amounts. An “effective amount” is that amount of an Arp2/3 inhibitor that alone, or together with further doses, produces the desired response, e.g. decreases expression or activity of an Arp2/3 molecule. The term “Arp2/3 inhibitor” is used synonymously with the terms “active compound”, “active agent” or active composition”. In the case of treating a subject having an injury, such as by a trauma, the desired response is increasing the nerve cell growth/motility and thus increasing the progression of the nerve regeneration. This may involve only a small increase in nerve function, although more preferably, it involves complete regeneration to produce a functioning neural connection. This can be monitored by routine methods or can be monitored according to diagnostic methods. The desired response to treatment of the disease or injury also can be delaying or preventing any worsening that may occur as a result of the disease or in jury or even preventing the onset of the disease.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of an Arp2/3 inhibitor for producing the desired response in a unit of weight or volume suitable for administration to a patient. The response can, for example, be measured by determining the effect on cell motility/neurite outgrowth following administration of the Arp2/3 inhibitor via a reporter system by measuring downstream effects such as increased cell motility motility/neurite outgrowth in vivo, or by isolating cells and measuring neural growth in vitro, or by measuring the physiological effects of the Arp2/3 inhibitor, or decrease of disease symptoms. Other assays will be known to one of ordinary skill in the art and can be employed for measuring the level of the response.

The doses of the active compounds (e.g., polypeptide, peptide, antibody, cell or nucleic acid) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of Arp2/3 inhibitor are formulated and administered in doses between 1 ng and 1 mg, and preferably between 10 ng and 100 μg, according to any standard procedure in the art. Where nucleic acids encoding an Arp2/3 inhibitor are employed, doses of between 1 ng and 0.1 mg generally will be formulated and administered according to standard procedures. Other protocols for the administration of Arp2/3 inhibitor will be known to one of ordinary skill in the art, in which the dose amount, schedule of injections, sites of injections, mode of administration and the like vary from the foregoing. Administration of Arp2/3 inhibitor to mammals other than humans, e.g. for testing purposes or veterinary therapeutic purposes, is carried out under substantially the same conditions as described above.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

An Arp2/3 inhibitor may be combined, if desired, with a pharmaceutically-acceptable carrier. The tern “pharmaceutically-acceptable carrier” as used herein means one or more compatible solid or liquid fillers, diluents or encapsulating substances which are suitable for administration into a human. The tern “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of Arp2/3 inhibitor, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

Screening assays for identifying potential drug candidates (and lead compounds) and determining the specificity's thereof are also provided according to the invention. For example, putative Arp2/3 binding compounds can be identified by screening libraries and the other methods described above. Knowing that a putative compound (peptide or non-peptide) interacts with Arp2/3 proteins, one can determine whether this compound is a drug that can exert a biological effect on nerve cell growth/migration, e.g., as an inhibitor of Arp2/3 by using any of the screening assays. The compounds which are identified using these screening assays are known as “lead” compounds. These lead compounds are then put through further testing, including, eventually, in vivo testing in animals and humans, from which the promise shown by the lead compounds in the original in vitro tests is either confirmed or refuted. See Remington's Pharmaceutical Sciences, 1990, A. R. Gennaro, ed., Chapter 8, pages 60-62, Mack Publishing Co., Easton, Pa.; Ecker and Crooke, 1995, Bio/Technology 13:351-360.

One example of a screening assay to identify lead compounds includes a method for identifying a therapeutic nerve growth promoter, by identifying a molecule capable of binding to Arp2/3 to identify a putative Arp2/3 binding molecule, contacting a nerve cell with the putative Arp2/3 binding molecule, and determining the effect of the putative Arp2/3 binding molecule on cell motility. The putative Arp2/3 binding molecule is a therapeutic nerve growth promoter when the nerve cell demonstrates enhanced motility with respect to a nerve cell that has not been contacted with the putative Arp2/3 binding molecule. The term “contacting” refers to the addition of a compound to an in vitro culture or the administration to a subject in vivo, such that the compound will taken up by a nerve cell.

### EXAMPLES

### Example 1

Introduction

To determine the pattern of expression of Arp2/3 in cells, the localization pattern of Arp2/3 in fibroblasts versus neurons was examined.

Methods

Rat2 fibroblasts and neuronal cells derived from the hippocampi of embryonic mice were infected with an adenovirus that drives the expression of a p21-EGFP (enhanced green fluorescent protein) fusion protein and analyzed using immunofluorescence microscopy to identify localization of Arp2/3 complex. p21-EGFP is an EGFP tagged version of p21, one of the subunits in the Arp2/3 complex.

Immunofluorescence Microscopy For immunofluorescence microscopy, cells were fixed in 4% paraformaldehyde, permeablized with 0.2% Triton X-100, and stained with texas-red phalloidin to visualize actin filaments. Coverslips were mounted using 4-Diazabicyclo-[2.2.2]Octane (DABCO) in 50% glycerol/10 mM Tris pH 8.0 and the EGFP and texas red staining visualized on a Zeiss Axiophote.

Results

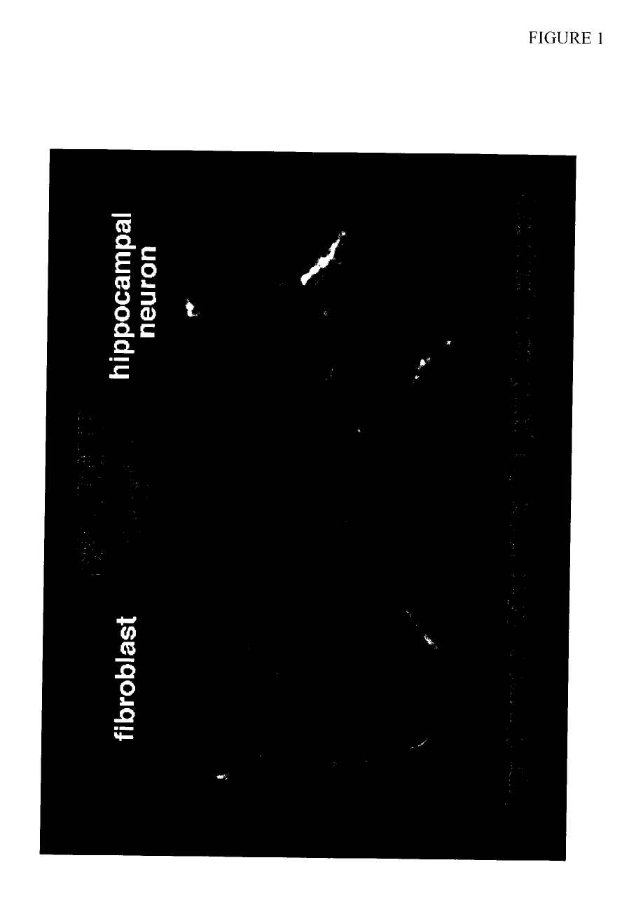

The distribution of Arp2/3 complex in fibroblast and nerve cells was analyzed by immunofluorescence (FIG. 1). The data demonstrate that the Arp2/3 (green) is localized at the edge of the fibroblasts. In contrast the Arp2/3 is localized in the central region of the hippocampal growth cone.

### Example 2

Introduction Several experiments were carried out to identify the role of Arp2/3 in neurons. The effects of an Arp2/3 inhibitor on neuronal growth cone translocation were examined using immunofluorescence.

Methods

Adenovirus Preparation

Recombinant adenoviruses were constructed using the pAdEASY system as described (He et al. (1998) PNAS 95:2509-14). Briefly, the sequence corresponding to amino acids 528-559 of bovine N-WASP (NWA) was cloned by PCR and fused in frame to the carboxy end of EGFP. The EGFP-NWA was then cloned into pShuttle-CMV and used to generate a recombinant adenovirus; pShuttle-CMV with EGFP alone was used as a control. Adenovirus was isolated from infected HEK-293 cells by freeze thaw, purified by CsCl banding, dialyzed into 25% glycerol/10 mM Tris pH 8.0/0.1% BSA, and stored in small aliquots at −80° C. Virus was titered by end point dilution in HEK293 cells.

Preparation and Infection of Cultures

Cultures were prepared from the hippocampi of embryonic day 16 (E16) Swiss Webster mice as previously described (Lanier et. al, (1999) Neuron 22:313-325.). Cultures were plated on poly-L-lysine coated coverslips and grown for 4 hrs in minimal essential medium (MEM), 10% horse serum, 0.6% glucose in 5% CO₂/37° C.

For stripe assays, poly-L-Lysine coated coverslips were stripped with purified Sema3A protein as described (Vielmetter et al. (1990) Exp. Brain Res. 81:283-287). After 4 hours, the medium was replaced with Neurobasal (Gibco) medium with B27 supplement (Gibco) that had been conditioned on post-natal cortical glial cells for two days. Cultures were then returned to the 5% CO₂/37° C. Approximately 24 hours after plating, recombinant adenoviruses were added to the culture medium at a multiplicity of infection (moi) of 50 and cultures were incubated for an additional 24-48 hrs.

At 24 or 48 hours after infection, the cultures were fixed with paraformaldehyde, processed for immunofluorescence, and examined on the microscope. Neurons were identified by staining for neuronal specific tubulin Beta-III. Cultures were fixed and visualized as previously described (Lanier et. al, (1999) Neuron 22:313-325.).

Results

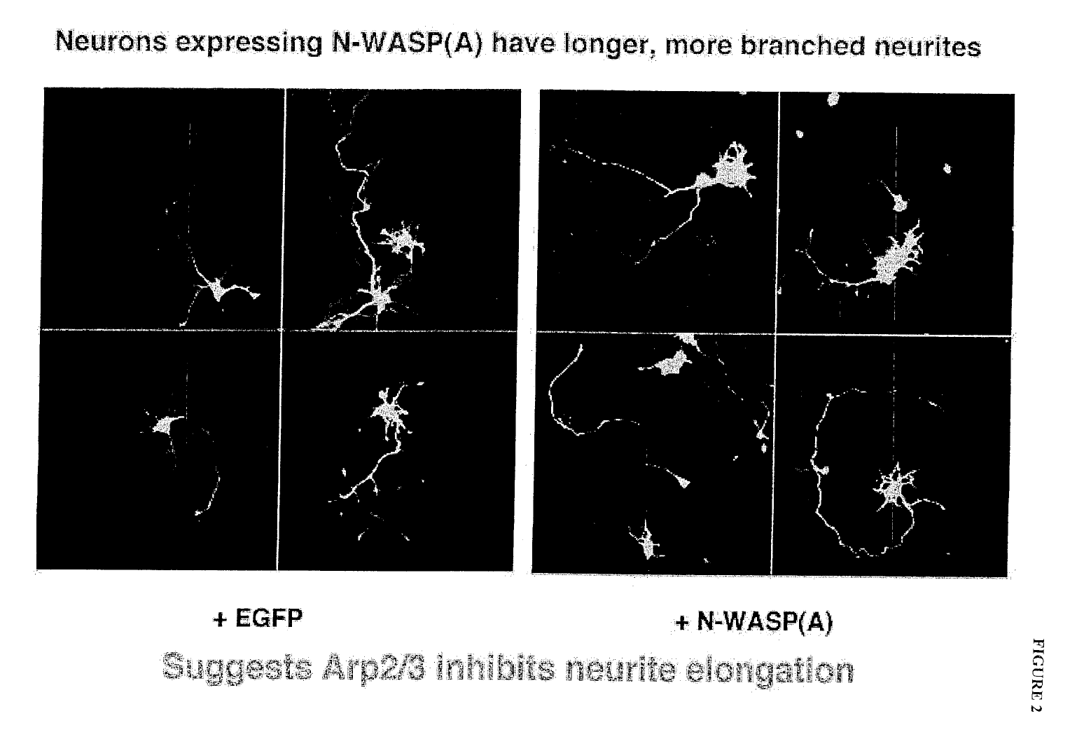

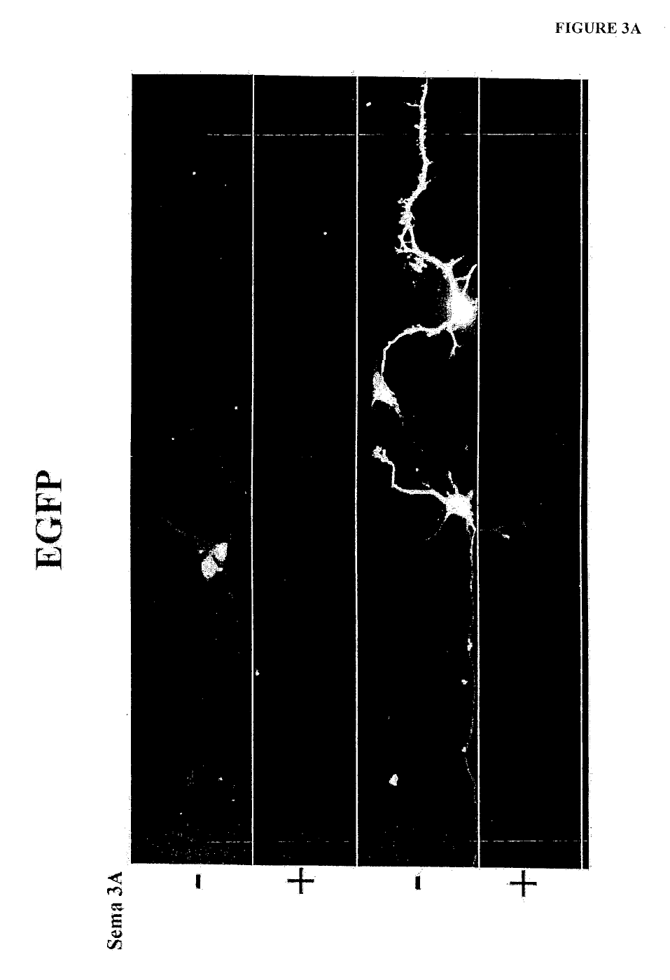

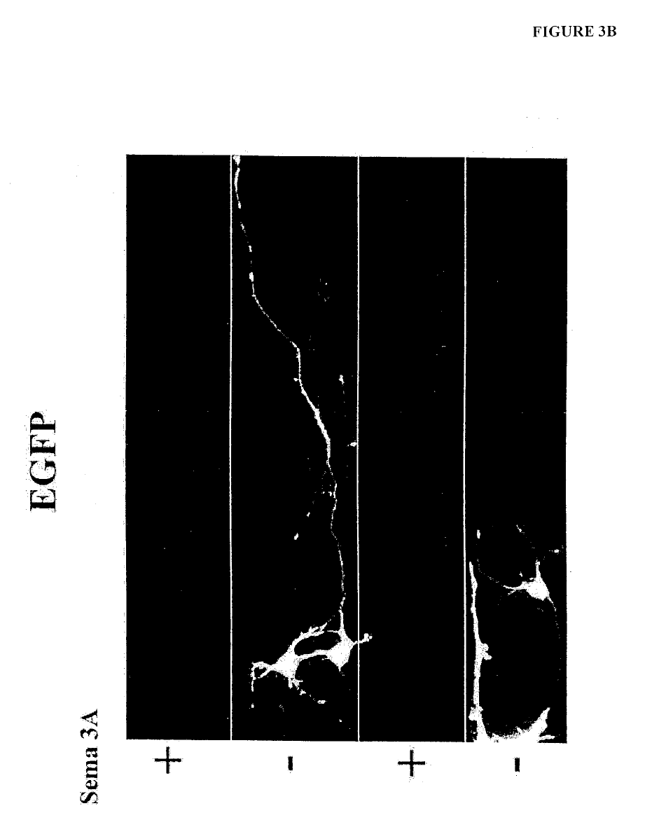

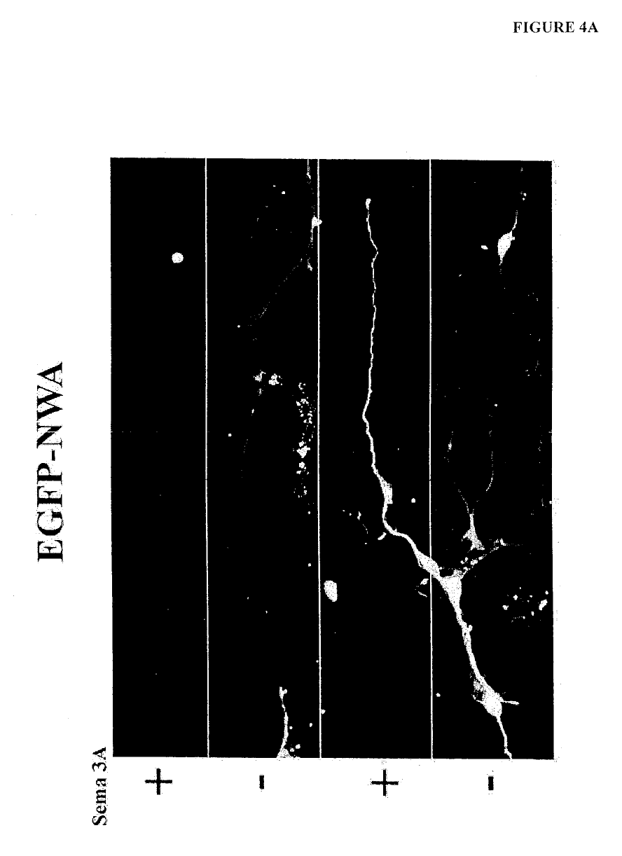

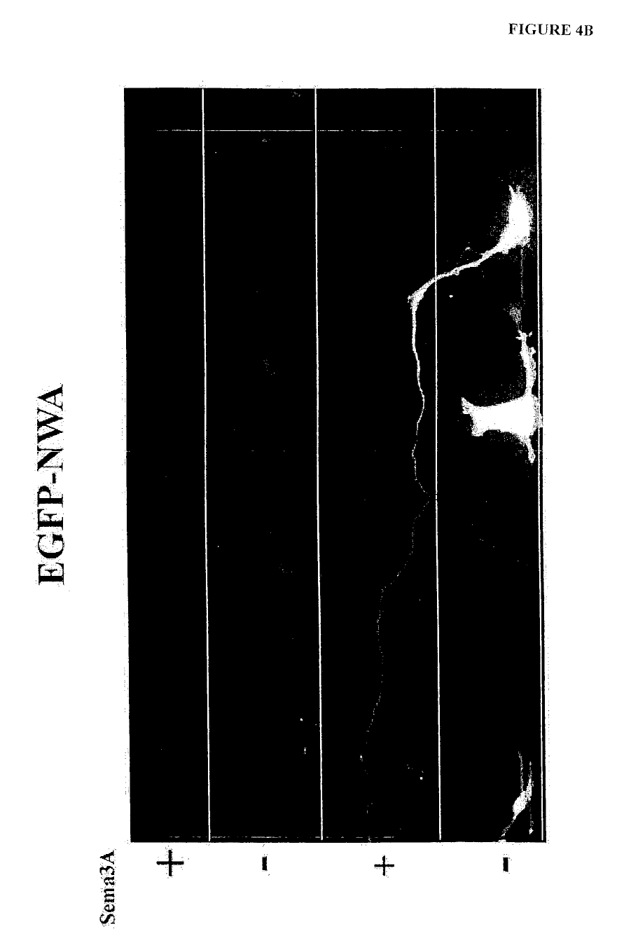

It was discovered that neurons expressing the EGFP-NWA construct had longer axons than controls that expressed EGFP alone (FIG. 2). To determine if axonal pathfinding was affected, neurons were plated on coverstips that had been stripped with Semaphorin 3A, an inhibitory guidance molecule. Cultures were infected and fixed as described above. Upon analysis, it was determined that neurons expressing the NWA construct frequently crossed the Semaphorin 3A stripes (FIGS. 4A and 4B), while the control neurons expressing EGFP alone rarely crossed the Semaphorin 3A stripe (FIGS. 3A and 3B). These findings indicate that expression of the EGFP-NWA construct enhances neurite outgrowth and enables the neurons to overcome inhibitory signals, indicating that the NWA construct are useful for promoting neural regeneration.

The foregoing written specification is considered to be sufficient to enable one skilled in the art to practice the invention. The present invention is not limited in scope by the examples provided, since the examples are intended as illustrations of various aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. All references, patents and patent publications that are recited in this application are incorporated in their entirety herein by reference.

We claim:

1. A method for enhancing nerve cell growth, comprising,
reducing Arp2/3 activity in a nerve cell in an effective amount to enhance nerve cell growth.

2. The method of claim 1, wherein the Arp2/3 activity is reduced by contacting the nerve cell with an Arp2/3 inhibitor.

3. The method of claim 2, wherein the Arp2/3 inhibitor is an Arp2/3 binding molecule that interacts with and reduces the activity of an Arp2/3 complex.

4. The method of claim 3, wherein the Arp2/3 binding molecule is an acidic domain of N-WASP or a functionally active fragment thereof.

5. The method of claim 4, wherein the Arp2/3 binding molecule is an acidic domain of WAVE or SCAR or a functionally active fragment thereof.

6. The method of claim 1, wherein the method is a method for promoting neural regeneration in vivo.

7. The method of claim 3, wherein the Arp2/3 binding molecule is a peptide mimetic.

8. The method of claim 2, wherein the Arp2/3 inhibitor is an Arp2/3 antisense molecule.

9. The method of claim 2, wherein the Arp2/3 inhibitor is an Arp2/3 RNAi molecule.

10. A method for identifying a therapeutic nerve growth promoter, comprising:
identifying a molecule capable of binding to Arp2/3 to identify a putative Arp2/3 binding molecule,
contacting a nerve cell with the putative Arp2/3 binding molecule, and
determining the effect of the putative Arp2/3 binding molecule on cell growth cone motility, wherein the putative Arp2/3 binding molecule is a therapeutic nerve growth promoter when the nerve cell demonstrates enhanced motility with respect to a nerve cell that has not been contacted with the putative Arp2/3 binding molecule.

11. A method for overcoming neuronal inhibitory signals, comprising:
contacting a nerve cell with an Arp2/3 inhibitor in an effective amount to overcome an inhibitory factor in the nerve cell.

12. A method for promoting nerve generation comprising,
contacting nerve cells with an Arp2/3 inhibitor in an effective amount to promote nerve generation.

13. The method of claim 12, wherein the Arp2/3 inhibitor is administered to a site of damaged nerve cells in a subject.

14. A method for treating neurodegeneration, comprising:
administering to a subject having or at risk of neurodegeneration an Arp2/3 inhibitor in an amount effective to treat neurodegeneration.

15. The method of claim 14, wherein the Arp2/3 inhibitor is administered locally to the site of neurodegeneration.

16. The method of claim 14, wherein the Arp2/3 inhibitor is administered to a nerve cell in vitro and the nerve cell is delivered to the subject at the site of neurodegeneration.

17. The method of claim 14, wherein the Arp2/3 inhibitor is administered in a sustained release vehicle at the site of neurodegeneration.

18. The method of claim 14, wherein the subject has or is at risk of developing a neurodegenerative disorder selected from the group consisting of Alzheimer's disease, Down Syndrome; Parkinson's disease; amyotrophic lateral sclerosis (ALS), stroke, direct trauma, Huntington's disease, epilepsy, ALS-Parkinsonism-dementia complex;
progressive supranuclear palsy; progressive bulbar palsy, spinomuscular atrophy, cerebral amyloidosis, Pick's atrophy, Retts syndrome; Wilson's disease, Striatonigral degeneration, corticobasal ganglionic degeneration; dentatorubral atrophy, olivo-pontocerebellar atrophy, paraneoplastic cerebellar degeneration; Tourettes syndrome, hypoglycemia; hypoxia; Creutzfeldt-Jakob disease; and Korsakoff's syndrome.
